(19) United States
(12) Patent Application Publication
(10) Pub. No.: US 2021/0223282 A1
(43) Pub. Date: Jul. 22, 2021

(54) METHOD AND DEVICE FOR RIGHT-LEFT DISCRIMINATION OF A GAIT TRAJECTORY

(71) Applicant: POU CHEN CORPORATION, Chang Hwa Hsien (TW)

(72) Inventor: Yu-Ping TSENG, Hsinchu County (TW)

(21) Appl. No.: 16/802,404

(22) Filed: Feb. 26, 2020

(30) Foreign Application Priority Data
Jan. 17, 2020 (TW) 109101693

(51) Int. Cl. G01P 13/00; G01P 15/18; G01P 1/00; G01C 19/5712; A61B 5/11; A61B 5/00

(52) U.S. Cl. CPC G01P 13/00; G01P 15/18; G01P 1/00; G01C 19/5712; A61B 5/0004; A61B 5/1122; A61B 5/6807; A61B 5/1114; A61B 5/112

(57) ABSTRACT

A method for right-left discrimination of a gait trajectory includes: a) obtaining a gait trajectory of a motion sensor based on motion information outputted by the motion sensor, where the motion sensor is mounted on one of left and right shoes, the motion information contains plural sets of coordinates representing positions of the one of left and right shoes, and the gait trajectory is constituted by the plural sets of coordinates; and b) calculating a slope of at least one line each between corresponding adjacent two sets of coordinates among the plural sets of coordinates, determining that the motion sensor is mounted on the right shoe When the slope is greater than zero, and determining that the motion sensor is mounted on the left shoe when the slope is smaller than zero.

### CROSS-REFERENCE TO RELATED APPLICATION

This application claims priority of Taiwanese Invention Patent Application No. 109101693, filed on Jan. 17, 2020.

### FIELD

The disclosure relates to a method and a device for gait analysis, and more particularly to a method and a device for right-left discrimination of a gait trajectory.

### BACKGROUND

A conventional smart insole, such as an intelligent insole module disclosed by Taiwanese Utility Model Patent Application No. M562025 or a conventional smart shoe, such as an intelligent shoes module disclosed by Taiwanese Utility Model Patent Application No. M562028, has a sensor unit disposed therein for collecting motion information.

The motion information is collected for a single insole or shoe, that is, the motion of the left foot and the motion of the right foot are sensed individually, and the motion information of the left shoe and the motion information of the right shoe are analyzed to monitor a gait of a user. In order to discriminate motion information collected by a sensor unit attached to the right shoe from motion information collected by a sensor unit attached to the left shoe during gait analysis, each of the sensor units is usually tagged in advance to indicate whether the motion information generated thereby relates to the left shoe or the right shoe. For example, a sensor unit tagged with a number “one” is mounted on a right shoe or a right sole, while a sensor unit tagged with a number “two” is mounted on a left shoe or a left sole. Whether the tag is realized by a physical approach or a software approach, the tag needs to be recognized first during assembly of the smart shoe or smart sole, making the manufacturing process more complex and troublesome.

### SUMMARY

Therefore, an object of the disclosure is to provide a method for right-left discrimination of a gait trajectory that can alleviate at least one drawback of the prior art and that can facilitate ease and convenience in the manufacturing process of a smart shoe.

A motion sensor is mounted on one of a left shoe and a right shoe. The motion sensor outputs motion information that contains plural sets of coordinates representing positions of the one of the left shoe and the right shoe. The method is to be implemented by a processing unit and includes:

a) obtaining a gait trajectory of the motion sensor based on the motion information outputted by the motion sensor, where the gait trajectory is constituted by the plural sets of coordinates; and

b) calculating a slope of at least one line each between corresponding adjacent two sets of coordinates among the plural sets of coordinates, determining that the motion sensor is mounted on the right shoe when it is determined that the slope is greater than zero, and determining that the motion sensor is mounted on the left shoe when it is determined that the slope is smaller than zero.

According to another aspect of the disclosure, a device is adapted for right-left discrimination of a gait trajectory. The device includes a motion sensor and a processing unit. The motion sensor is to be mounted on one of a left shoe and a right shoe and is configured to output motion information that contains plural sets of coordinates representing positions of the one of the left shoe and the right shoe. The processing unit includes a processor configured to obtain a gait trajectory of the motion sensor based on the motion information outputted by the motion sensor, where the gait trajectory is constituted by the plural sets of coordinates. The processor is further configured to calculate a slope of at least one line each between corresponding adjacent two sets of coordinates among the plural sets of coordinates, to determine that the motion sensor is mounted on the right shoe when it is determined that the slope is greater than zero, and to determine that the motion sensor is mounted on the left shoe when it is determined that the slope is smaller than zero.

### BRIEF DESCRIPTION OF THE DRAWINGS

Other features and advantages of the disclosure will become apparent the following detailed description of the embodiments with reference to the accompanying drawings, of which:

FIG. 1 is a perspective view illustrating an embodiment of a device for right-left discrimination of a gait trajectory according to the disclosure;

FIG. 2 is a block diagram illustrating the embodiment of the device according to the disclosure;

FIG. 3 is a perspective view illustrating a variation of the embodiment where a processing unit is integrated. in an external electronic device;

FIG. 4 is a flow chart illustrating an embodiment of a method for right-left discrimination of a gait trajectory according to the disclosure;

FIG. 5 is a schematic diagram illustrating, for each of left and right shoes, plural sets of coordinates constituting the gait trajectory; and

FIG. 6 is a schematic diagram illustrating, for each of the left and right shoes, a slope of a line defined between adjacent two sets of coordinates.

### DETAILED DESCRIPTION

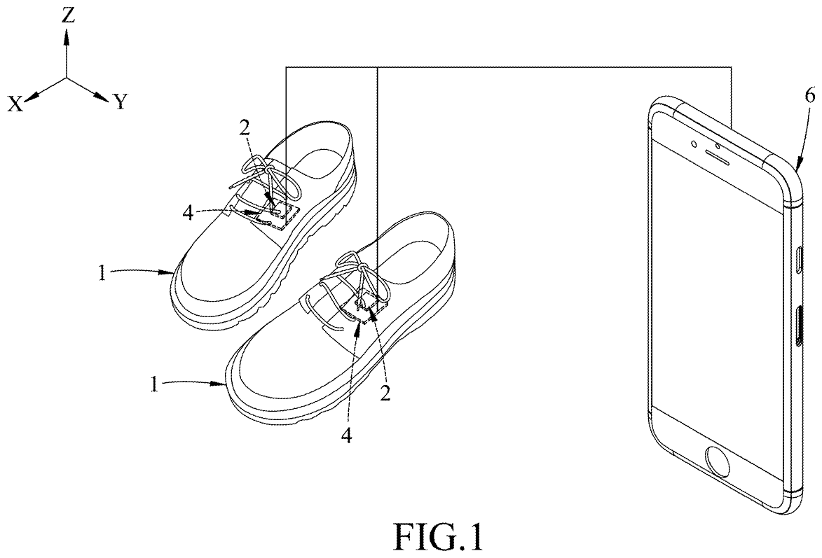

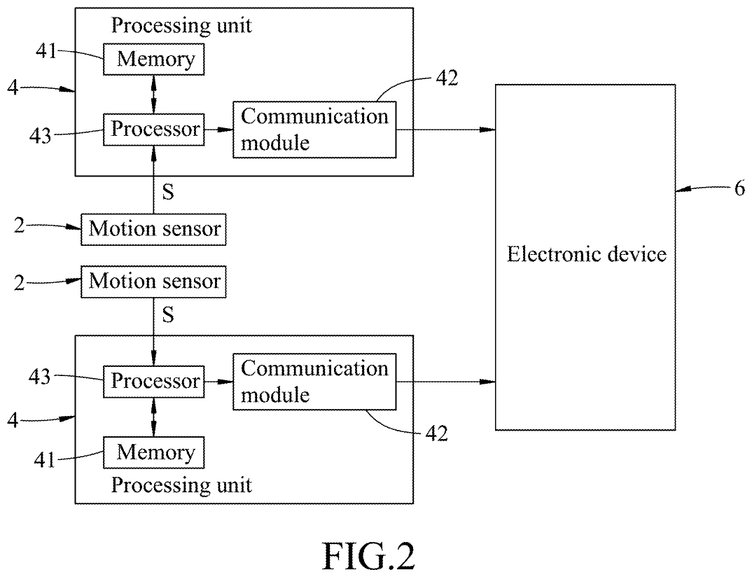

Referring to FIGS. 1 and 2, an embodiment of a device for right-left discrimination of a gait trajectory according to the disclosure is illustrated. The device is to be mounted on a pair of shoes 1, i.e., left and right shoes, worn on the left foot and the right foot of a user. The device includes two motion sensors 2 and two processing units 4.

Each of the motion sensors 2 is to be mounted on a respective one of the left and right shoes, and is configured to output motion information (S). The motion information (S) contains plural sets of coordinates (P) representing positions of the respective one of the left and right shoes. In this embodiment, each of the motion sensors 2 is disposed in a midsole of the respective one of the left and right shoes, and is disposed adjacent to the arch of the foot of the user. However, in other embodiments, the motion sensors 2 may be disposed in the outsoles or attached to outside surfaces of the shoes 1.

In this embodiment, each of the motion sensors 2 is implemented by micro-electro-mechanical systems (MEMS) and includes one of a three-axis accelerometer which detects acceleration on three axes, a gyroscope which detects angular speed on the three axes, and a combination thereof. The motion information (S) contains the plural sets of coordinates (P) in a Cartesian coordinate system defined by an X-axis parallel to a direction in which the user is progressing straight when wearing the shoes 1, a Y-axis perpendicular to the X-axis and located on a horizontal plane, and a Z-axis perpendicular to the X-axis and the Y-axis. It is noted that in this embodiment, only the coordinates on the X-axis and the Y-axis are taken into account when making the right-left discrimination of a gait trajectory.

Each of the processing units 4 is to be mounted on a respective one of the left and right shoes, and is electrically connected to one of the motion sensors 2 that is mounted on the respective one of the left and right shoes. Each of the processing units 4 includes a memory 41 for storing data, a communication module 42 for communication with other devices through a set of wireless communication technologies, and a processor 43 electrically connected to the memory 41 and the communication module 42. The set of wireless communication technologies includes, but not limited to, one of Bluetooth, Wi-Fi, Narrowband Internet of Things (NB-IoT), enhanced Machine Type Communication (eMTC), Long-Term Evolution, category M1 (LTE Cat-M1), and any combination thereof.

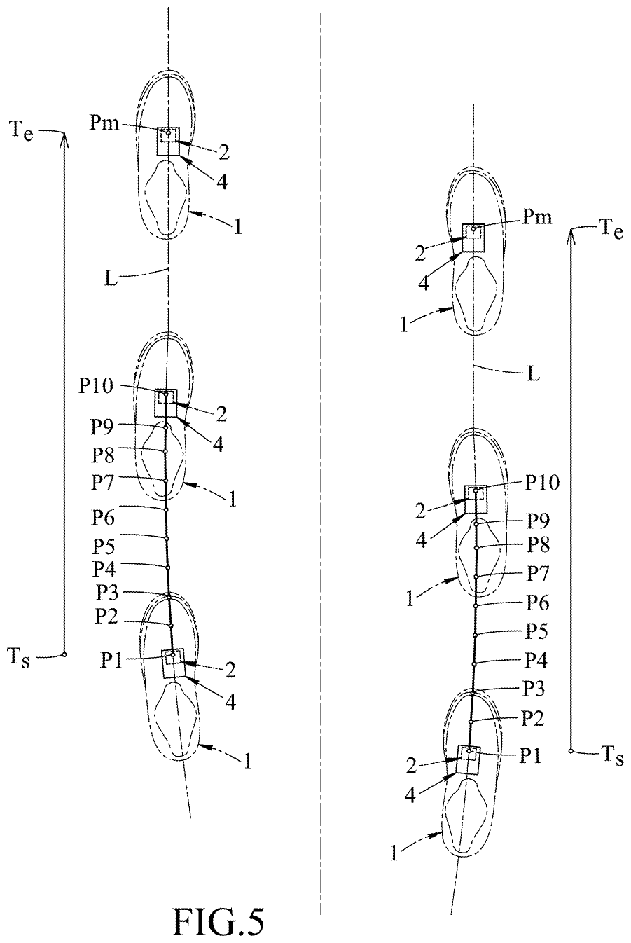

Referring to FIGS. 2 and 5, for each of the motion sensors 2, the motion information (S) outputted during movement of the motion sensor 2 contains plural sets of coordinates (P), and the processor 43 of the respective one of the processing units 4 obtains the plural sets of coordinates (P) based on the motion information (S) outputted by the motion sensor 2. Based on the plural sets of coordinates (P), a moving distance, a direction and an angle of the respective one of the shoes 1 may be calculated for subsequent gait analysis. Since the three-axis accelerometer and the gyroscope are well known to a person having ordinary skill in the art, detailed descriptions on implementation of the same are omitted herein for the sake of brevity.

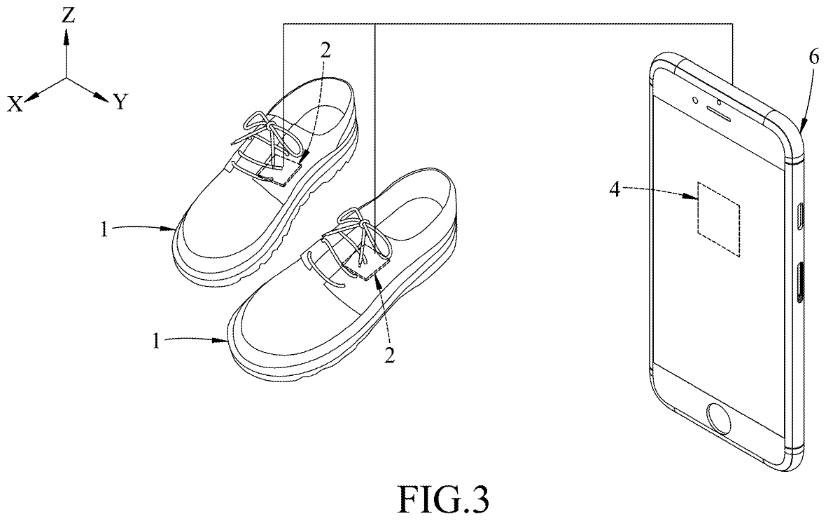

It is noted that each of the processing units 4 is communicable via the communication module 42 with an external electronic device 6 that is located at a remote location, such as a smart phone, a tablet computer or a notebook computer. In addition, the number of the processing units 4 is not limited to two. Referring to FIG. 3, in a variation of this embodiment, there is only one processing unit 4, and the processing unit 4 is separated from the shoes 1 and integrated in the electronic device 6.

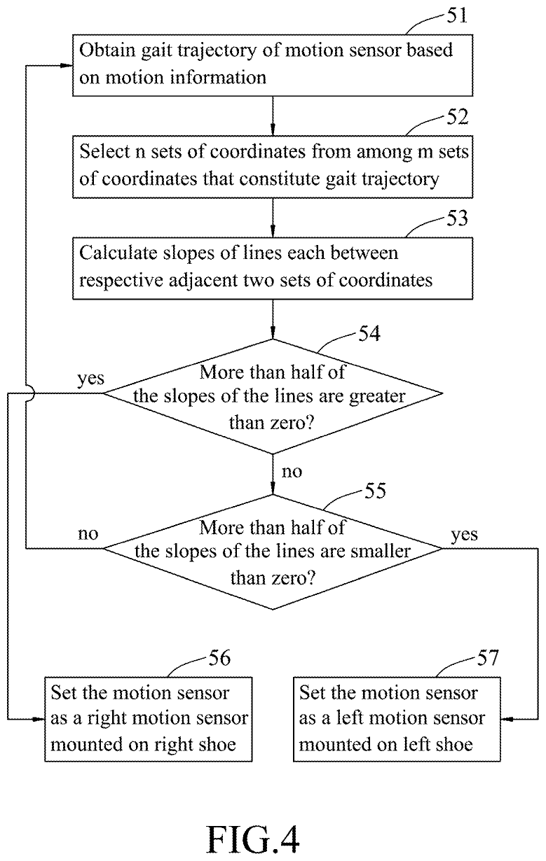

Referring to FIGS. 1, 2, 4 and 5, an embodiment of a method for right-left discrimination of a gait trajectory according to the disclosure is illustrated. The method is to be implemented by the processor 43 of each of the processing units 4. It is noted that only one of the shoes 1 and the motion sensor 2 and the processing unit 4 mounted on said one of the shoes 1 are discussed in the following steps for the sake of clear explanation of the method. However, it is readily understandable that the following steps are also applicable to the motion sensor 2 and the processing unit 4 on the other of the shoes 1.

In step 51, the processor 43 obtains a gait trajectory (L) of the motion sensor 2 during movement of the shoe 1 based on the motion information (S) outputted by the motion sensor 2 within a time period between a start time point (Tₛ) and an end time point (Tₑ). In this embodiment, the time period between the start time point (Tₛ) and the end time point (Tₑ) ranges from one second to three seconds. The number of sets of coordinates (P) obtained by the processor 43 based on the motion information (S) outputted by the motion sensor 2 within the time period is m, where m ranges between 10 and 50. The gait trajectory (L) is constituted by the in sets of coordinates (P) corresponding to the time period.

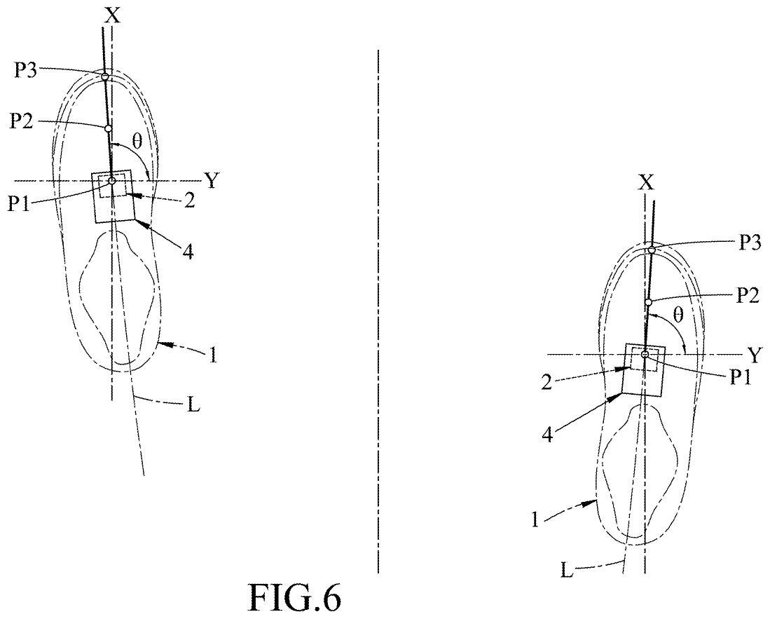

In step 52, the processor 43 selects n sets of coordinates (P) from among the m sets of coordinates constituting the gait trajectory (L), where any adjacent two sets of coordinates among the n sets of coordinates define a line. In this embodiment, n is ten. Furthermore, the n sets of coordinates are selected by the processor 43 sequentially from a first set to an n^{th} set, and the first set of coordinates (P) is obtained by the processor 43 at the start time point (Tₛ). In other words, the processor 43 selects the first n sets of coordinates (P) in step 52. For ease of understanding, the first to the n^{th} (i.e., tenth in this embodiment) set of coordinates (P) are denoted as P1 to P10 in FIGS. 5 and 6.

It is noted that in this embodiment, the gait trajectory (L) corresponds to one stride of the user. However, in other embodiments, the gait trajectory (L) may correspond to two to five strides of the user, and n is not limited to ten. The greater the number of strides monitored and the greater the number “n”, the more accurate the result of the right-left discrimination.

In step 53, the processor 43 calculates slopes of the lines each between respective adjacent two sets of coordinates among the n sets of coordinates. The slopes of the lines are calculated relative to the Y-axis.

In step 54, the processor 43 makes a determination as to whether at least one of the slopes of the lines is greater than zero. More specifically, the processor 43 determines whether more than half of the slopes of the lines are greater than zero, that is, whether more than five of the lines have slopes greater than zero.

Referring to FIG. 6, whether or not the slope of a line is greater than zero is decided by an included angle (θ) between the line and the Y-axis. The slope is calculated according to a formula, k=tan θ, where k represents the slope. When the included angle (θ) is smaller than 90 degrees, the slope is greater than zero, and when the included angle (θ) is greater than 90 degrees, the slope is smaller than zero.

In step 55, the processor 43 makes another determination as to whether at least one of the slopes of the lines is smaller than zero when a result of the determination made in step 54 is negative. More specifically, the processor 43 determines whether more than half of the slopes of the lines are smaller than zero, that is, whether more than five of the lines have slopes smaller than zero.

When the result of the determination made in step 54 is positive, in step 56, the processor 43 sets the motion sensor 2 as a right motion sensor mounted on the right shoe.

When a result of the another determination made in step 55 is positive, in step 57, the processor 43 sets the motion sensor 2 as a left motion sensor mounted on the left shoe. Otherwise, when the result of the another determination made in step 55 is negative, a flow of this method goes back to step 51.

In this way, the processor 43 of the processing unit 4 is able to automatically determine whether the motion sensor 2 mounted on one of the shoes 1 is a right motion sensor mounted on the right shoe or a left motion sensor mounted on the left shoe, so as to realize the right-left discrimination of a gait trajectory. After the right-left discrimination is made, the processor 43 is able to analyze gait of the left foot and right foot, such as out-toeing or in-toeing, left deviation or right deviation from the midline, eversion or inversion, forefoot strike or rearfoot strike, and so forth.

To sum up, the processing unit 4 of this disclosure is able to automatically determine whether a motion sensor 2 is mounted on the left shoe or the right shoe. As a result, the motion sensors 2 are not required to be tagged in advance to distinguish right from left. Modularization of the motion sensors 2 may be achieved. Ease and convenience in assembling the motion sensors 2 and the shoes 1 may also be promoted. Moreover, the result of the right-left discrimination may be utilized in subsequent gait analysis, helping to improve accuracy of the gait analysis.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for right-left discrimination of a gait trajectory, a motion sensor mounted on one of a left shoe and a right shoe, the motion sensor outputting motion information that contains plural sets of coordinates representing positions of the one of the left shoe and the right shoe, the method to be implemented by a processing unit and comprising:
a) obtaining a gait trajectory of the motion sensor based on the motion information outputted by the motion sensor, where the gait trajectory is constituted by the plural sets of coordinates; and
b) calculating a slope of at least one line each between corresponding adjacent two sets of coordinates among the plural sets of coordinates, determining that the motion sensor is mounted on the right shoe when it is determined that the slope is greater than zero, and determining that the motion sensor is mounted on the left shoe when it is determined that the slope is smaller than zero.

2. The method for right-left discrimination of a gait trajectory as claimed in claim 1, wherein step b) includes sub-steps of:
b-1) selecting n sets of coordinates from among the plural sets of coordinates constituting the gait trajectory, where any adjacent two sets of coordinates among the n sets of coordinates define a line;
b-2) calculating slopes of the lines each between respective adjacent two sets of coordinates among the n sets of coordinates;
b-3) making a determination as to whether at least one of the slopes of the lines is greater than zero;
b-4) setting the motion sensor as a right motion sensor mounted on the right shoe when a result of the determination made in step b-3) is positive;
b-5) making another determination as to whether at least one of the slopes of the lines is smaller than zero when the result of the determination made in step b-3) is negative;
b-6) setting the motion sensor as a left motion sensor mounted on the left shoe when a result of the another determination made in step b-5) is positive; and
b-7) returning to step a) when the result of the another determination made in step b-5) is negative.

3. The method for right-left discrimination of a gait trajectory as claimed in claim 2, wherein:
in step b-3), the processing unit makes the determination as to whether more than half of the slopes of the lines are greater than zero; and
in step b-5), the processing unit makes the another determination as to whether more than half of the slopes of the lines are smaller than zero.

4. The method for right-left discrimination of a gait trajectory as claimed in claim 2, wherein step a) includes:
obtaining the gait trajectory based on the motion information which is outputted by the motion sensor within a time period between a start time point and an end time point, where the time period ranges from one second to three seconds.

5. The method for right-left discrimination of a gait trajectory as claimed in claim 4, further comprising:
obtaining m sets of coordinates based on the motion information outputted by the motion sensor within the time period between the start time point and the end time point, where m ranges between 10 and 50, the gait trajectory being constituted by the m sets of coordinates.

6. The method for right-left discrimination of a gait trajectory as claimed in claim 4, wherein in step b-1), then sets of coordinates are selected by the processing unit sequentially from a first one of the plural sets of coordinates, and the first one of the plural sets of coordinates is obtained by the processing unit at the start time point.

7. A device for right-left discrimination of a gait trajectory comprising:
a motion sensor to be mounted on one of a left shoe and a right shoe and configured to output motion information that contains plural sets of coordinates representing positions of the one of the left shoe and the right shoe; and
a processing unit including a processor configured to
obtain a gait trajectory of said motion sensor based on the motion information outputted by said motion sensor, where the gait trajectory is constituted by the plural sets of coordinates,
calculate a slope of at least one line each between corresponding adjacent two sets of coordinates among the plural sets of coordinates,
determine that said motion sensor is mounted on the right shoe when it is determined that the slope is greater than zero, and
determine that said motion sensor is mounted on the left shoe when it is determined that the slope is smaller than zero.

8. The device for right-left discrimination of a gait trajectory as claimed in claim 7, wherein said device comprises two of said motion sensors, and two of said processing units electrically and respectively connected to said motion sensors.

9. The device for right-left discrimination of a gait trajectory as claimed in claim 7, wherein said processing unit is communicable with said motion sensor through a set of wireless communication technologies.

10. The device for right-left discrimination of a gait trajectory as claimed in claim 7, wherein any adjacent two sets of coordinates among the plural sets of coordinates define a line, and said processor obtains the gait trajectory based on the motion information which is outputted by said motion sensor within a time period between a start time point and an end time point, where the time period ranges from one second to three seconds.
